(19) United States
(12) Patent Application Publication
(10) Pub. No.: US 2026/0263731 A1
(43) Pub. Date: Sep. 10, 2026

(54) SLEEP MASK FOR PEOPLE WITH INSOMNIA

(71) Applicant: IMAM ABDULRAHMAN BIN FAISAL UNIVERSITY, Dammam (SA)

(72) Inventors: Maryam AL OTHMAN, Dammam (SA); Hala EL-WAKEEL, Dammam (SA); Noran KATTAN, Dammam (SA); Khadijah AL KHUWAITEM, Dammam (SA); Amal ALKHAYYAL, Dammam (SA)

(73) Assignee: IMAM ABDULRAHMAN BIN FAISAL UNIVERSITY, Dammam (SA)

(21) Appl. No.: 19/071,630

(22) Filed: Mar. 5, 2025

(51) Int. Cl. A61M 21/02; A61F 9/04; A61N 5/06; A61M 21/00

(52) U.S. Cl. CPC A61M 21/02; A61F 9/045; A61N 5/0618; A61M 2021/0044; A61M 2021/0083; A61M 2021/0088; A61M 2205/8206; A61M 2207/00; A61N 2005/0626; A61N 2005/063; A61N 2005/0648; A61N 2005/0651; A61N 2005/0663

(57) ABSTRACT

A sleep mask and a method of making thereof are described. The sleep mask includes sound generating assemblies positioned at opposite sides of an eye shield, each assembly comprising a programmable memory card, printed circuit board, microprocessor, rechargeable lithium ion battery, and speaker installed within an ear cushion inner ring cavity. The memory cards store first, second, and third sound patterns, voice tracks, and illumination parameters. Upon activation, the first sound pattern generates breathing guidance sounds for a predetermined duration, followed by the second sound pattern producing white noise until deactivation or sleep time expiration, whereupon the third sound pattern generates a wake-up alarm. The breathing guidance sounds are synchronized with the voice tracks and the illumination patterns. An elastic head band secure the eye shield and sound generating assemblies while maintaining optimal positioning during use.

### BACKGROUND

### TECHNICAL FIELD

The present disclosure is directed to sleep aids and sleeping masks, and more particularly to a sleep mask with integrated sound generation capabilities for guiding breathing patterns and providing white noise to improve sleep quality for users suffering from insomnia.

### DESCRIPTION OF RELATED ART

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Sleep disorders, particularly insomnia, represent a significant health challenge affecting a large portion of the adult population. Studies indicate that approximately 56.9% of adults with sleep disorders suffer from insomnia, with the condition being particularly prevalent among professionals aged 20-40 years who experience high workplace stress. Insomnia is characterized by difficulty falling asleep or maintaining continuous sleep, leading to inadequate rest and reduced daytime productivity. Traditional approaches to treating insomnia have primarily focused on pharmaceutical interventions or basic sleep accessories that provide limited functionality. While conventional sleep masks can block ambient light, they fail to address the underlying physiological and psychological factors that contribute to sleep difficulties, such as irregular breathing patterns and racing thoughts that often prevent sleep onset.

Current sleep aid devices on the market typically offer singular functions, such as simple light blocking or basic white noise generation. Some advanced devices incorporate audio capabilities but lack integration with breathing guidance or fail to provide a comprehensive solution that addresses multiple aspects of sleep initiation and maintenance. Existing solutions also tend to be either overly simplistic, missing critical features needed for effective sleep assistance, or too complex, requiring external devices or complicated setup procedures that can interfere with the sleep preparation process. Additionally, many current devices lack the proper ergonomic design necessary for comfortable all-night wear while maintaining consistent audio delivery. Furthermore, power management and control interfaces in current devices often present operational challenges.

US20150092972A1 describes headwear incorporating an audio delivery device and microprocessor for playing sounds such as white noise. The headwear includes eye flaps and an accelerometer for monitoring sleep patterns, with capabilities to provide sound and light cues during sleep periods. However, this reference is audio delivery headgear for playing sounds and is not particularly directed to inducing sleep, the light cues are not patterns of light emitted to induce and prolong sleep, nor are the sounds a pattern of breathing sounds followed by white noise meant to induce sleep.

US20220240016A1 describes a wearable electronic device with ear openings and speakers, utilizing fabric layers with stiffened edge portions to hold electronics. The device may stretches across the face to cover the eyes and includes speakers for playing white noise or sleep-oriented audio tracks. However, this reference does not detail the sleep-oriented sound tracks.

US20210375253A1 describes an ear and eye mask with an ear covering comprising an outer cup and passive noise-excluding member defining a cavity, with a speaker placed within the aural cavity. However, this reference does not describe a pattern of breathing sounds followed by white noise meant to induce sleep.

The Aura smart sleep mask provides sound, light, and alarm functionality through Bluetooth connectivity, played via an external device, offering guided meditations and nature soundscapes. However, this device does not include integrated circuitry programmed with a pattern of breathing sound followed by white noise meant to induce sleep.

Each of the aforementioned references suffers from one or more drawbacks hindering their adoption, such as inconsistent audio delivery, poor ergonomic design for extended wear, lack of integrated therapeutic approaches, and inability to maintain proper positioning throughout sleep cycles. Many existing solutions require multiple separate components or complex setup procedures that can interfere with the sleep preparation process. Additionally, current devices often fail to effectively combine audio guidance, comfort, and proper anatomical fit in a single integrated solution. Accordingly, it is one object of the present disclosure to provide a sleep mask that overcomes these limitations through a design that integrates multiple therapeutic approaches while maintaining user comfort and operational simplicity.

### SUMMARY

In an exemplary embodiment, a sleep mask is described, comprising: an eye shield configured to conform to a shape of a nose and eyes of a user; a first sound generating assembly including a first speaker; a second sound generating assembly including a second speaker, wherein each sound generating assembly includes a printed circuit board, an ear cushion configured with an inner ring cavity and a cover, wherein the cover is configured to interlock with the ear cushion to enclose the printed circuit board, wherein the first speaker is configured to fit into the inner ring cavity of the ear cushion of the first sound generating assembly and the second speaker is configured to fit into the inner ring cavity of the ear cushion of the second sound generating assembly; and an elastic head band including a first segment connected to a proximal edge of the ear cushion of the first sound generating assembly and a second segment connected to a distal edge of the ear cushion of the first sound generating assembly, a third segment connected to a proximal edge of the ear cushion of the second sound generating assembly and a fourth segment connected to a distal edge of the ear cushion of the second sound generating assembly, wherein the elastic head band is configured to hold the eye shield over the nose and eyes of the user and the sound generating assembly over each ear of the user.

In another exemplary embodiment, a method of making a sleep mask is described, comprising: programming a first memory card with a first sound pattern, a second sound pattern, a third sound pattern, a voice track, a first pattern of colors, a second pattern of colors and a state of charge threshold; attaching the first memory card to a first printed circuit board including a first microprocessor; attaching a first rechargeable lithium ion battery to the first microprocessor; installing a first speaker within an inner ring cavity of a first ear cushion; forming a first sound generating assembly by attaching a first cover over the first ear cushion, the first speaker, the first memory card, the first printed circuit board, the first microprocessor and the first rechargeable lithium ion battery, wherein the first microprocessor includes one or more processors configured to execute the instructions to generate the sound patterns and transmit the sound patterns to the first speaker; programming a second memory card with the first sound pattern, the second sound pattern, the third sound pattern, the voice track, the first pattern of colors, the second pattern of colors and the state of charge threshold; attaching the second memory card to a second printed circuit board including a second microprocessor; attaching a second rechargeable lithium ion battery to the second microprocessor; installing a second speaker within an inner ring cavity of a second ear cushion; forming a second sound generating assembly by attaching a second cover over the second ear cushion, the second speaker, the second memory card, the second printed circuit board, the second microprocessor and the second rechargeable lithium ion battery, wherein the second microprocessor includes one or more processors configured to execute the instructions to generate the sound patterns and transmit the sound patterns to the second speaker; forming an eye shield configured to conform to a shape of a nose and eyes of a user; connecting a first end of a first elastic head band to a first end of the eye shield; connecting a second end of the first elastic head band to the first cover; and connecting a first end of a second elastic head band to a second end of the eye shield; connecting a second end of the second elastic head band to the second cover, wherein the first elastic head band and the second elastic head band configured to hold the eye shield over the nose and eyes of the user, the first sound generating assembly over a first ear of the user and the second sound generating assembly over a second ear of the user.

The foregoing general description of the illustrative embodiments and the following detailed description thereof are merely exemplary aspects of the teachings of this disclosure and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A is an exemplary perspective view of a sleep mask, according to certain embodiments.

FIG. 1B is an exemplary front elevational view of the sleep mask, according to certain embodiments.

FIG. 1C is an exemplary side elevational view of the sleep mask, according to certain embodiments.

FIG. 1D is an exemplary top elevational view of the sleep mask, according to certain embodiments.

FIG. 1E is an exemplary exploded perspective diagram of the sleep mask from a first side thereof, according to certain embodiments.

FIG. 1F is an exemplary exploded perspective diagram of the sleep mask from a second side thereof, according to certain embodiments.

FIG. 2A is an exemplary illustration of the sleep mask being worn by a user prior to activation, according to certain embodiments.

FIG. 2B is an exemplary illustration of the sleep mask in operation, including a representative audio waveform pattern generated by a sound generating assembly thereof, according to certain embodiments.

FIG. 3 is an exemplary sequence diagram illustrating breathing guidance provided by the sleep mask, including inhale, hold, and exhale phases with corresponding durations, according to certain embodiments.

FIG. 4 is an exemplary flowchart listing steps involved in a method of manufacturing the sleep mask, according to certain embodiments.

FIG. 5 is an illustration of a non-limiting example of details of computing hardware used in the computing system, according to certain embodiments.

FIG. 6 is an exemplary schematic diagram of a data processing system used within the computing system, according to certain embodiments.

FIG. 7 is an exemplary schematic diagram of a processor used with the computing system, according to certain embodiments.

FIG. 8 is an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a”, “an” and the like generally carry a meaning of “one or more”, unless stated otherwise.

Furthermore, the terms “approximately,” “approximate”, “about” and similar terms generally refer to ranges that include the identified value within a margin of 20%, 10%, or preferably 5%, and any values therebetween.

The term “proximal” in the present disclosure refers to a position close to the head.

The term “distal” in the present disclosure refers to a position spaced from or pointing away from the head.

Aspects of this disclosure are directed to a sleep mask and a method of making a sleep mask which provide comprehensive sleep assistance through an integrated approach to sleep therapy. The sleep mask of the present disclosure describes technologies for combining various sleep-inducing elements into a unified, comfortable design that addresses multiple aspects of sleep initiation and maintenance. The present sleep mask particularly focuses on providing consistent audio delivery while maintaining anatomical conformity and user comfort throughout extended sleep periods.

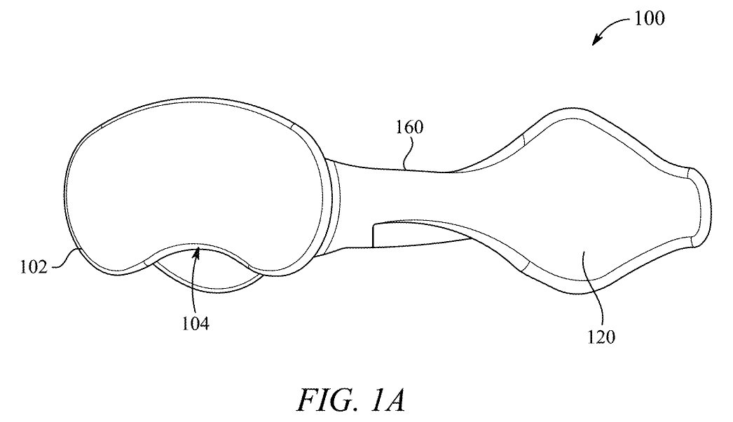

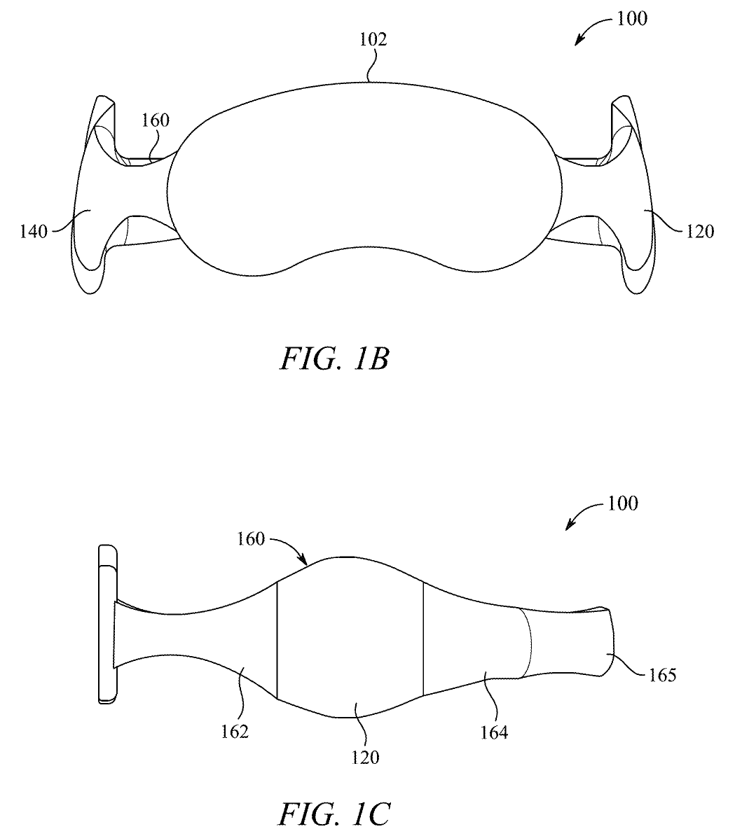

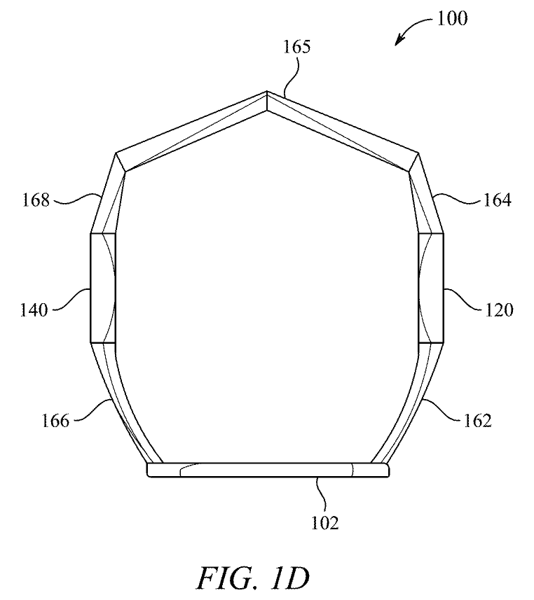

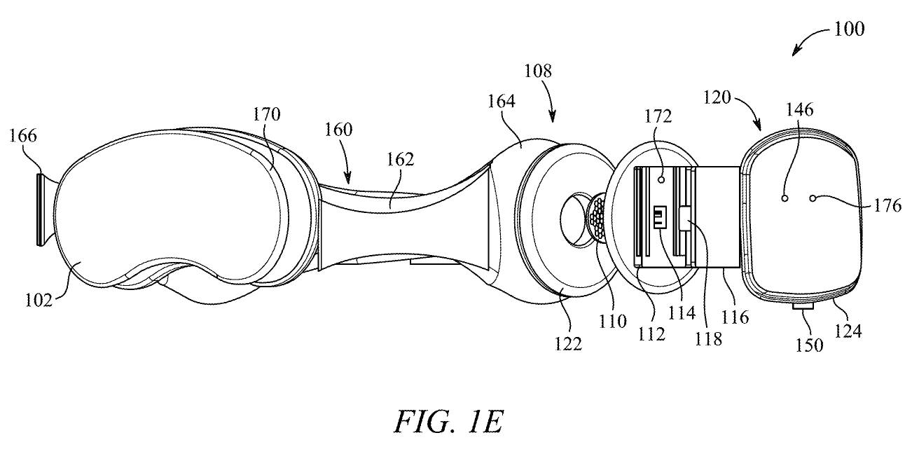

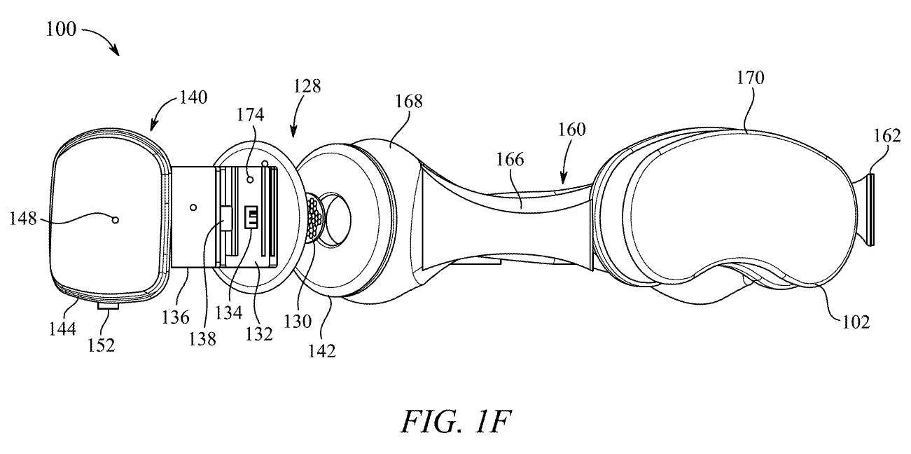

Referring to FIGS. 1A-1F in combination, illustrated are different views of a sleep mask (as represented by reference numeral 100). Herein, FIG. 1A illustrates a perspective view of a sleep mask 100; FIG. 1B illustrates a front elevational view of the sleep mask 100; FIG. 1C illustrates a side elevational view of the sleep mask 100; FIG. 1D illustrates a top elevational view of the sleep mask 100; FIG. 1E illustrates an exploded perspective diagram of the sleep mask 100 from a first side thereof; and FIG. 1F illustrates an exploded perspective diagram of the sleep mask 100 from a second side thereof. The sleep mask 100 of the present disclosure enables customized sleep assistance through programmable therapeutic sequences, making it particularly suitable for users with varying sleep requirements, including those with irregular schedules or stress-induced sleep difficulties. The sleep mask 100 is designed for sleep therapy through its multi-component configuration. The sleep mask 100 integrates various therapeutic elements into a unified system, allowing for synchronized delivery of different sleep-inducing therapies while maintaining optimal positioning and comfort, and addressing multiple aspects of sleep quality improvement.

The sleep mask 100 is designed for both functional effectiveness and user comfort, incorporating features to create an optimal sleep environment. The design of the sleep mask 100 considers the full spectrum of sleep-related needs, from initial relaxation through maintaining sleep quality to gentle awakening, all while ensuring consistent therapeutic delivery throughout the sleep cycle. This approach makes the sleep mask 100 especially valuable for professionals in high-stress environments where sleep quality directly impacts performance and well-being. The sleep mask 100 may be used to serve various applications, from regular nightly use to travel situations where sleep conditions may be suboptimal.

As illustrated, the sleep mask 100 includes an eye shield 102 configured to conform to a shape of a nose and eyes of a user. The eye shield 102 incorporates specific anatomical contours designed to accommodate various facial features while maintaining consistent positioning throughout sleep cycles. Herein, the eye shield 102 includes a contoured lower edge 104 to accommodate the nose bridge of the user. The contoured lower edge 104 comprises a curved recess portion dimensioned to rest against the sides of the nose without applying excessive pressure. The contoured lower edge 104 incorporates reinforced stitching along its perimeter to maintain structural integrity while preventing material fraying or deterioration. The specific geometry of the contoured lower edge 104 is designed based on an analysis to accommodate a wide range of facial structures.

In an aspect, the eye shield 102 is made of a layer of dust mite, mold, and mildew resistant cotton covered by a fade resistant stretchable silk satin fabric. In particular, the eye shield 102 includes a layered construction incorporating a base layer of dust mite, mold, and mildew resistant cotton material serving as the primary structural component. The base layer of the eye shield 102 is covered by a fade-resistant stretchable silk satin fabric providing both aesthetic finishing and additional comfort properties. Silk satin is made of silk fibers. As used herein, the term “silk satin” refers to a material that is made from silk fibers and has been weaved using the satin technique. Satin is a weave that uses a 4:1 ratio, which means there are 4 weft threads wrapped under 1 warp thread. In this case, the weave is performed using silk fibers. Such layered construction of the eye shield 102 provides enhanced light blocking capabilities while maintaining breathability during extended wear periods.

As shown in FIG. 1E, the sleep mask 100 further includes a first sound generating assembly 108 positioned on a first side of the eye shield 102, configured to provide audio output to a first ear of the user. The first sound generating assembly 108 includes multiple integrated components housed within a unified structure. The first sound generating assembly 108 includes a first speaker 110, a first printed circuit board 112 incorporating a microprocessor 114, a first rechargeable lithium ion battery 116 (rated at 3000mAh capacity), and a first programmable memory card 118 (with about 12GB storage capacity) programmed with specific sound patterns. The first sound generating assembly 108 also includes a first ear cushion 120 configured with a first inner ring cavity 122 and a first cover 124. These components are arranged in a specific configuration within the first sound generating assembly 108 in consideration of both acoustic performance and weight distribution.

In the first sound generating assembly 108, the first ear cushion 120 is fabricated from a cotton material selected for both comfort and acoustic properties. Herein, the first speaker 110 is configured to fit into the first inner ring cavity 122 of the first sound generating assembly 108. The first inner ring cavity 122 is specifically dimensioned to house the first speaker 110 while providing acoustic isolation and maintaining user comfort. The first inner ring cavity 122 of the first ear cushion 120 is configured to create an enclosed acoustic chamber that enhances sound delivery while preventing external noise interference. The first cover 124 is configured to interlock with the first ear cushion 120 to enclose the printed circuit board 112 and other internal electronic components. In an example, the first cover 124 is fabricated from polyethylene (PE) plastic material. The first cover 124 may also include interlocking features that engage with corresponding features on the first ear cushion 120 to create a secure enclosure for the electronic components. In an aspect, the interlocking features may include posts on either side of the speaker support (shown between the speaker 130 and the printed circuit board 112 in FIG. 1E) and pressure fit post reception holes in the first inner ring cavity 122 and the inner surface of the cover 124. In another aspect, the interlocking features may simply include patches hook and loop fasteners (for example, Velcro^{TM}, placed on the surface of the first inner ring cavity, on either side of the speaker support and on the inner surface of the cover 124, which would allow the ear cushion assembly to be opened to replace the rechargeable lithium ion battery 116 as needed.

Herein, the first rechargeable lithium ion battery 116 is connected to the first printed circuit board 112. The first rechargeable lithium ion battery 116 may be physically and electrically connected to the first printed circuit board 112 through conductive traces and mounting points configured on the first printed circuit board 112. The connection between the first rechargeable lithium ion battery 116 and the first printed circuit board 112 enables power distribution to all electronic components within the first sound generating assembly 108. The first programmable memory card 118 is connected to the first printed circuit board 112, through a connector on the first printed circuit board 112. The first programmable memory card 118 includes program instructions stored in non-volatile memory which include sound patterns. The first microprocessor 114 is connected to the first printed circuit board 112, the first programmable memory card 118, the first rechargeable lithium ion battery 116, and the first speaker 110. The first microprocessor 114 is mounted directly on and electrically connected to the first printed circuit board 112 through conductive pathways. The first microprocessor 114 maintains electrical connections to the first programmable memory card 118 through data and control lines, to the first rechargeable lithium ion battery 116 through power management circuitry and to the first speaker 110 through audio signal lines.

In the first sound generating assembly 108, the first microprocessor 114 includes one or more processors configured to execute the instructions to generate the sound patterns and transmit the sound patterns to the first speaker 110. Specifically, the one or more processors execute the program instructions stored in the first programmable memory card 118 to generate the sound patterns according to predefined sequences. The first microprocessor 114 processes these sound patterns and transmits the processed audio signals to the first speaker 110 for acoustic output. The first microprocessor 114 of the first sound generating assembly 108 is also configured to coordinate with the second sound generating assembly 128 to ensure synchronized audio output from the first speaker 110 and the second speaker 130.

The sleep mask 100 further includes a second sound generating assembly 128 positioned on a second side of the eye shield 102, configured to provide audio output to a second ear of the user, as shown in FIG. 1F. The second sound generating assembly 128 includes multiple integrated components housed within a unified structure. The second sound generating assembly 128 includes a second speaker 130, a second printed circuit board 132 incorporating a second microprocessor 134, a second rechargeable lithium ion battery 136 (rated at 3000mAh capacity), and a second programmable memory card 138 (with about 12GB storage capacity) programmed with specific sound patterns. The second sound generating assembly 128 also includes a second ear cushion 140 configured with a second inner ring cavity 142 and a second cover 144. These components are arranged in a specific configuration within the second sound generating assembly 128 in consideration of both acoustic performance and weight distribution.

In the second sound generating assembly 128, the second ear cushion 140 includes cotton material selected for both comfort and acoustic properties. Herein, the second speaker 130 is configured to fit into the second inner ring cavity 142 of the second ear cushion 140 of the second sound generating assembly 128. The second inner ring cavity 142 is specifically dimensioned to house the second speaker 130 while providing acoustic isolation and maintaining user comfort. The second inner ring cavity 142 of the second ear cushion 140 is configured to create an enclosed acoustic chamber that enhances sound delivery while preventing external noise interference. The second cover 144 is configured to interlock with the second ear cushion 140 to enclose the printed circuit board 132 and other internal electronic components as described with respect to the first ear cushion assembly of FIG. 1E. In an example, the second cover 144 is fabricated from polyethylene (PE) plastic material. The second cover 144 may also include interlocking features that engage with corresponding features on the second ear cushion 140 to create a secure enclosure for the electronic components.

Herein, the second rechargeable lithium ion battery 136 is connected to the second printed circuit board 132. The second rechargeable lithium ion battery 136 may be physically and electrically connected to the second printed circuit board 132 through conductive traces and mounting points configured on the second printed circuit board 132. The connection between the second rechargeable lithium ion battery 136 and the second printed circuit board 132 enables power distribution to all electronic components within the second sound generating assembly 128. The second programmable memory card 138 is connected to the second printed circuit board 132, through a connector or the like mounted on the second printed circuit board 132. The second programmable memory card 138 includes program instructions stored in non-volatile memory which include sound patterns. The second microprocessor 134 is connected to the second printed circuit board 132, the second programmable memory card 138, the second rechargeable lithium ion battery 136, and the second speaker 130. The second microprocessor 134 is mounted directly on and electrically connected to the second printed circuit board 132 through conductive pathways. The second microprocessor 134 maintains electrical connections to the second programmable memory card 138 through data and control lines; to the second rechargeable lithium ion battery 136 through power management circuitry; and to the second speaker 130 through audio signal lines.

In the second sound generating assembly 128, the second microprocessor 134 includes one or more processors configured to execute the instructions to generate the sound patterns and transmit the sound patterns to the second speaker 130. Specifically, the one or more processors execute the program instructions stored in the second programmable memory card 138 to generate the sound patterns according to predefined sequences. The second microprocessor 134 processes these sound patterns and transmits the processed audio signals to the second speaker 130 for acoustic output. The second microprocessor 134 of the second sound generating assembly 128 is also configured to coordinate with the first sound generating assembly 108 to ensure synchronized audio output between the second speaker 130 and the first speaker 110.

Further, in the sleep mask 100, specific materials are selected for various components based on their mechanical and electrical properties. Herein, the cover 124, 144, the memory card 118, 138 and an enclosure of the speaker 110, 130 of each sound generating assembly 108, 128 respectively are made of polyethylene and each printed circuit board 112, 132 is made of polychlorinated biphenyl plastic. The first cover 124 and the second cover 144 are fabricated from the polyethylene (PE) plastic material selected for durability and electrical insulation properties. The first programmable memory card 118 and the second programmable memory card 138 utilize polyethylene-based circuit substrates. The enclosures for the first speaker 110 and the second speaker 130 incorporates polyethylene construction for optimal acoustic properties and environmental protection. The first printed circuit board 112 and the second printed circuit board 132 are constructed using polychlorinated biphenyl plastic material, selected due to its electrical insulation properties and resistance to low temperatures.

The sleep mask 100 further includes a first indicator light 146 located on the first cover 124 of the first sound generating assembly 108 and a second indicator light 148 located on the second cover 144 of the second sound generating assembly 128. In the first sound generating assembly 108, the first microprocessor 114 incorporates integrated battery monitoring circuitry configured to determine a state of charge of the first rechargeable lithium ion battery 116. The first microprocessor 114 executes a monitoring routine that compares the determined state of charge to a predetermined threshold value stored in the first programmable memory card 118. When the state of charge falls below this threshold value, the first microprocessor 114 generates a control signal to illuminate the first indicator light 146. Similarly, in the second sound generating assembly 128, the second microprocessor 134 determines a state of charge of the second rechargeable lithium ion battery 136, compares the state of charge to the threshold value stored in the second programmable memory card 138, and controls illumination of the second indicator light 148 based on the comparison.

The sleep mask 100 further includes a first charge jack 150 connected to the cover of the first sound generating assembly 108 and a second charge jack 152 connected to the cover of the second sound generating assembly 128, for recharging purposes. The first charge jack 150 is connected to the first cover 124 of the first sound generating assembly 108 through a dedicated mounting aperture. The first charge jack 150 includes electrical contacts configured to interface with a shared charger (not shown) for recharging the first rechargeable lithium ion battery 116 of the first sound generating assembly 108. The second charge jack 152 is physically connected to the second cover 144 of the second sound generating assembly 128 through a similar mounting aperture. The second charge jack 152 includes electrical contacts configured to interface with the shared charger for recharging the second rechargeable lithium ion battery 136 of the second sound generating assembly 128. The shared charger is designed to provide appropriate charging voltage and current levels for battery maintenance and longevity.

The sleep mask 100 further includes an elastic head band 160 configured for secure positioning and user comfort during extended wear periods. The elastic head band 160 has a multi-segment design comprising four distinct segments for optimal tension distribution and positioning stability. A first segment 162 of the elastic head band 160 connects to a proximal edge of the first ear cushion 120 of the first sound generating assembly 108, while a second segment 164 connects to a distal edge of the first ear cushion 120. The elastic head band 160 further includes a third segment 166 connected to a proximal edge of the second ear cushion 140 of the second sound generating assembly 128, and a fourth segment 168 connected to a distal edge of the second ear cushion 140. The elastic head band 160 is configured to hold the eye shield 102 over the nose and eyes of the user and the sound generating assembly 108, 128 over each ear of the user. Herein, the elastic head band 160 helps to maintain specific tension characteristics that enable consistent positioning of both the eye shield 102 and the sound generating assemblies 108, 128 during sleep cycles. The multi-segment configuration of the elastic head band 160 ensures the eye shield 102 remains properly positioned over the nose and eyes of the user while simultaneously maintaining optimal acoustic positioning of the first sound generating assembly 108 and the second sound generating assembly 128 over the respective ears of the user. Each segment 162, 164, 166, 168 of the elastic head band 160 incorporates connection points specifically utilized for secure attachment to the ear cushions 120, 140 through reinforced stitching patterns or the like.

As illustrated, the elastic head band 160 may also incorporate an adjustable fastening mechanism 165 positioned at the posterior junction of the second segment 164 and the fourth segment 168. The fastening mechanism 165 may be in the form of a hook and loop closure system in which a hook component extends from a terminal portion of the second segment 164 and a corresponding loop component extends from a terminal portion of the fourth segment 168. The hook and loop closure components are dimensioned to provide approximately 5 centimeters of adjustable overlap, enabling circumferential adjustment of the elastic head band 160 to accommodate various head circumferences while maintaining optimal tension distribution. The fastening mechanism 165 provides for secure engagement between the second segment 164 and the fourth segment 168 through multiple points of mechanical interlocking between the hook and loop surfaces, providing reliable retention force while facilitating adjustability for user comfort and proper positioning of the sound generating assemblies 108, 128 relative to the ears of the user.

The sleep mask 100 further includes a fiber optic fabric 170 integrated within both the eye shield 102 and the elastic head band 160. The fiber optic fabric 170 comprises a network of optical fibers positioned to provide uniform illumination distribution. A first light emitting diode 172 is located on the first printed circuit board 112 of the first sound generating assembly 108, and a second light emitting diode 174 is located on the second printed circuit board 132 of the second sound generating assembly 128. The first light emitting diode 172 is electrically connected to the first microprocessor 114 and optically coupled to a first plurality of optical fibers (not shown) in the fiber optic fabric 170. Similarly, the second light emitting diode 174 is electrically connected to the second microprocessor 134 and optically coupled to a second plurality of optical fibers (not shown) in the fiber optic fabric 170. The first microprocessor 114 and the second microprocessor 134 are configured to actuate the respective light emitting diodes 172, 174 to illuminate the fiber optic fabric 170 specifically in colors within the red spectrum. This illumination is synchronized with the sound patterns to enhance sleep during operation of the first sound pattern, the second sound pattern, or simultaneous operation of both sound patterns.

In particular, the first microprocessor 114 of the first sound generating assembly 108 executes specific control algorithms to drive the first light emitting diode 172 according to a first frequency pattern, thereby generating a first pattern of colors within the red spectrum. Concurrently, the second microprocessor 134 of the second sound generating assembly 128 drives the second light emitting diode 174 according to a second frequency pattern, generating a second pattern of colors within the red spectrum. The fiber optic fabric 170 is configured such that the first pattern of colors and the second pattern of colors combine and interact within the optical fibers, producing a new color pattern. This new color pattern is specifically configured through the mixing of the two distinct frequency patterns to generate wavelengths demonstrated to enhance sleep quality. The timing and intensity parameters of both frequency patterns are programmed into the respective memory cards 118, 138 and are coordinated by the microprocessors 114, 134 to maintain optimal illumination characteristics throughout the sleep cycle.

The sleep mask 100 further includes a power button 176 located on the first cover 124 of the first sound generating assembly 108. The power button 176 is connected to the first rechargeable lithium ion battery 116 and the first microprocessor 114 of the first sound generating assembly 108. Specifically, the power button 176 is electrically connected to both the first rechargeable lithium ion battery 116 and the first microprocessor 114. When actuated, the power button 176 initiates a power-on sequence in the first microprocessor 114, which then begins executing the programmed sound pattern sequences. In the present configuration, the power button 176 may incorporate tactile feedback elements to confirm successful actuation while maintaining the water-resistant properties of the first cover 124. The placement of the power button 176 is specifically engineered to allow easy access during use while preventing accidental activation.

In the sleep mask 100, the first microprocessor 114 of the first sound generating assembly 108 serves as the primary control unit for audio generation and synchronization. The first microprocessor 114 is configured to generate the sound patterns and transmit the sound patterns to the first speaker 110 of the first sound generating assembly 108 and to the second speaker 130 of the second sound generating assembly 128. The first microprocessor 114 generates the programmed sound patterns according to stored sequences and transmit these patterns simultaneously to both the first speaker 110 of the first sound generating assembly 108 and the second speaker 130 of the second sound generating assembly 128. Herein, the first speaker 110 of the first sound generating assembly 108 and the second speaker 130 of the second sound generating assembly 128 generate the sound patterns in tandem. For this purpose, the first speaker 110 and the second speaker 130 are calibrated, ensuring coherent audio output for therapeutic effect. The timing parameters for the tandem operation are stored in the first programmable memory card 118 and executed by the first microprocessor 114 to maintain audio synchronization throughout all operating modes.

In the sleep mask 100, the sound patterns generated by each sound generating assembly 108, 128 includes a first sound pattern, a second sound pattern and a third sound pattern. The first sound pattern is configured to generate breathing sounds to guide a sequence of breaths of the user for a first period of time when the power button 176 is turned ON. The first sound pattern is automatically initiated when the power button 176 transitions to the ON state. The first sound pattern comprises a precisely timed sequence of audio signals to guide the breathing rhythm of the user. The second sound pattern is configured to generate white noise after the first period of time until one of the power button 176 is turned OFF and a programmed sleep time has expired. The second sound pattern activates automatically upon completion of the first sound pattern. The second sound pattern generates calibrated white noise to mask ambient sounds and maintain sleep continuity. The white noise generation continues either until manual deactivation through the power button 176 or until reaching a pre-programmed sleep duration threshold stored in the programmable memory cards 118, 138. The third sound pattern is configured to generate a wake-up alarm sound when the programmed sleep time has expired. This pattern generates a wake-up alarm sequence with gradually increasing volume to provide gentle arousal from sleep. The third sound pattern is coordinated between both sound generating assemblies 108, 128 to maintain balanced stereo output during the wake-up sequence. The specific acoustic characteristics and timing parameters of the wake-up alarm are stored within the programmable memory cards 118, 138 and executed by the microprocessors 114, 134 according to user-defined sleep duration settings.

In an aspect, the first sound pattern includes a sequence of repetitious breathing sounds configured to repeat three times every five minutes. Herein, the sequence of repetitious breathing sounds includes a breath inhaling sound generated for about four seconds, a breath holding sound generated for about seven seconds and a breath exhaling sound generated for about eight seconds. That is, the first sound pattern implements a precise respiratory guidance protocol through repetitious breathing sequences. Each complete breathing cycle spans nineteen seconds and consists of three distinct audio phases: a breath inhaling sound spanning four seconds (±0.1s), followed by a breath holding sound maintained for seven seconds (±0.1s), concluding with a breath exhaling sound extending for eight seconds (±0.1s). This complete sequence repeats three times within each five-minute interval, with timing controlled by the microprocessors 114, 134 to maintain consistent respiratory guidance.

Herein, the first sound pattern of each sound generating assembly 108, 128 further includes a voice track configured to provide counting sounds as the sequence of repetitious breathing sounds are generated. The first sound pattern incorporates an integrated voice track synchronized with the breathing sounds. The voice track generates numerical counting sounds that correspond to each phase of the breathing sequence. During the four-second inhalation phase, the voice track counts “one, two, three, four,” followed by counting through the seven-second hold phase, and concluding with an eight-count exhale sequence. The microprocessors 114, 134 maintain synchronization between the breathing sounds and the voice track throughout each repetition. In an aspect, the first period of time is about twenty minutes. The first period of time, during which the first sound pattern operates, is specifically programmed to span twenty minutes (±30s). This duration accommodates about twelve complete breathing cycles, with four sets of three-sequence repetitions.

In an aspect, the first sound generating assembly 108 further includes a set of alarm control buttons (not shown) located on the first cover 124 and connected to the first microprocessor 114. Herein, the set of alarm control buttons may include an hour adjustment button configured to increment hour values, a minute adjustment button configured to increment minute values in predefined intervals, and a set button configured to confirm programmed times. Each button in the set of alarm control buttons incorporates tactile feedback elements. The first sound generating assembly 108 further includes a clock timer (not shown) located within the first microprocessor 114. The clock timer is integrated within the architecture of the first microprocessor 114 and provides both a real-time clock function for current time maintenance and a separate alarm time register for wake-up programming. Herein, the set of alarm control buttons are configured to program the first microprocessor 114 to generate the wake-up alarm sound of the third sound pattern. For this purpose, when the user activates the alarm control buttons, the first microprocessor 114 enters a programming mode where user inputs are captured and stored in the alarm time register of the clock timer. Once programmed, the clock timer continuously compares the current time against the stored alarm time. When these times match, the first microprocessor 114 initiates the wake-up alarm sound sequence of the third sound pattern. The wake-up timing parameters are stored within the first programmable memory card 118 and executed according to the user-programmed settings.

The sleep mask 100 incorporates specific dimensional parameters optimized for user comfort and functional effectiveness. In an example configuration, the eye shield 102 has a width of approximately 19 centimeters and a height of approximately 7 centimeters, dimensioned to provide complete coverage of the eye area while maintaining a compact form factor. Each sound generating assembly 108, 128 extends approximately 5 centimeters from the side edges of the eye shield 102, with the ear cushions 120, 140 having a diameter of approximately 7 centimeters to ensure proper acoustic seal around the ears. The elastic head band 160 has an adjustable circumference ranging from approximately 52 centimeters to 62 centimeters to accommodate various head sizes, with each segment 162, 164, 166, 168 having a width of approximately 2 centimeters for optimal pressure distribution. The overall depth of the sleep mask 100 from the front surface of the eye shield 102 to the back surface of the sound generating assemblies 108, 128 is approximately 9 centimeters.

The sleep mask 100 utilizes specific materials selected for durability, comfort, and functional performance. The eye shield 102 incorporates a multi-layer construction, with an outer layer comprising fade-resistant silk satin fabric and an inner layer of hypoallergenic cotton material. The elastic head band 160 utilizes medical-grade elastic material with enhanced durability properties, maintaining elasticity through repeated use cycles. The sound generating assemblies 108, 128 incorporate impact-resistant polyethylene plastic for the covers 124, 144, with the ear cushions 120, 140 utilizing memory foam material wrapped in breathable cotton fabric. The printed circuit boards 112, 132 are constructed using flame-retardant FR-4 glass-reinforced epoxy laminate material, providing thermal stability and electrical insulation properties. The speakers 110, 130 utilize neodymium magnets with copper voice coils for optimal acoustic performance while maintaining low weight.

The sleep mask 100 incorporates additional features for enhanced functionality and user experience. The eye shield 102 includes ventilation channels configured within the cotton padding to facilitate air circulation and prevent heat buildup during extended wear periods. The sound generating assemblies 108, 128 include waterproof membrane covers protecting the electronic components from moisture exposure. The programmable memory cards 118, 138 include secure digital card interfaces enabling user-customizable sound pattern uploads through standard card readers. The rechargeable lithium ion batteries 116, 136 incorporate overcharge protection circuitry and thermal monitoring capabilities. The elastic head band 160 includes silicone grip sections on the inner surface to prevent slippage during sleep movements. The covers 124, 144 incorporate raised tactile indicators adjacent to control buttons for improved accessibility in low-light conditions.

In operation, the sleep mask 100 executes a programmed sequence of therapeutic functions initiated through actuation of the power button 176. Upon activation, the first microprocessor 114 of the first sound generating assembly 108 transmits a wireless signal to the second microprocessor 134 to turn ON and pair with the first microprocessor 114. The first microprocessor 114 initiates the first sound pattern comprising breathing guidance sequences. The first sound pattern generates a timed sequence consisting of a four-second breath inhaling sound, followed by a seven-second breath holding sound, and concluded with an eight-second breath exhaling sound. This complete nineteen-second breathing sequence repeats three times within each five-minute interval over a twenty-minute duration. The first microprocessor 114 also synchronizes the audio output between the first speaker 110 of the first sound generating assembly 108 and the second speaker 130 of the second sound generating assembly 128 in a pairing relationship.

Following completion of the first sound pattern, the first microprocessor 114 generates the second sound pattern comprising calibrated white noise. The second sound pattern continues either until manual deactivation through the power button 176 or until reaching a pre-programmed sleep duration threshold stored within the first programmable memory card 118 and the second programmable memory card 138. Upon reaching the programmed sleep duration threshold, the first microprocessor 114 initiates the third sound pattern comprising a wake-up alarm sequence. The third sound pattern implements gradually increasing volume levels coordinated between the first speaker 110 and the second speaker 130 to provide audio output during the wake-up sequence. The sleep mask 100 can be deactivated at any point during operation through actuation of the power button 176, which triggers an orderly shutdown sequence in the first microprocessor 114.

During operation, the first light emitting diode 172 and the second light emitting diode 174 illuminate the fiber optic fabric 170 according to synchronized frequency patterns generating wavelengths within the red spectrum demonstrated to enhance sleep quality. The first microprocessor 114 maintains timing of all operational sequences through the clock timer, comparing current time against stored alarm time parameters to ensure accurate execution of the wake-up alarm functionality. Further during operation, the first microprocessor 114 monitors the state of charge of the first rechargeable lithium ion battery 116 through integrated battery monitoring circuitry. When the monitored state of charge falls below a predetermined threshold value stored in the first programmable memory card 118, the first microprocessor 114 generates a control signal to illuminate the first indicator light 146. Similarly, the second microprocessor 134 performs battery monitoring for the second rechargeable lithium ion battery 136, controlling illumination of the second indicator light 148 based on the monitored charge state.

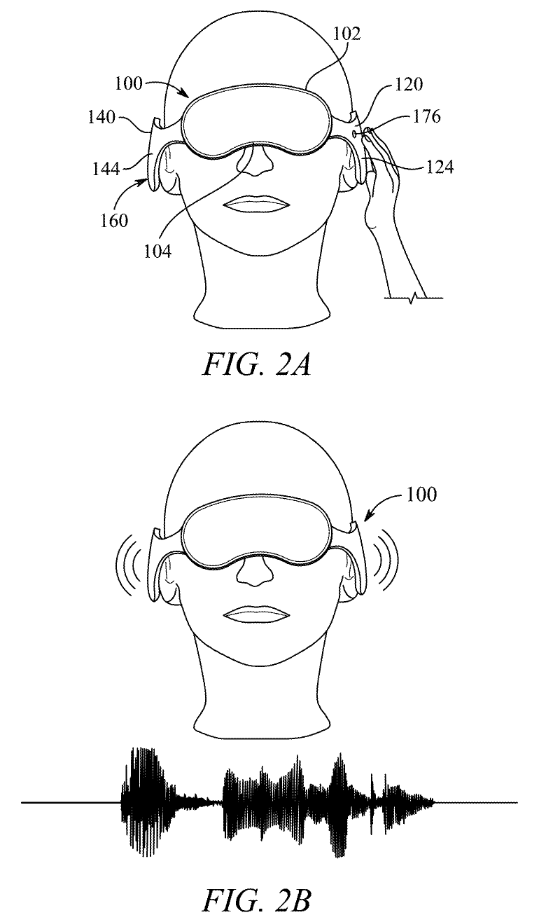

Referring to FIG. 2A, illustrated is the sleep mask 100 positioned on a head of a user in a pre-activation state. The eye shield 102 is shown conforming to the facial contours of the user, with the contoured lower edge 104 accommodating the nose bridge region. The first sound generating assembly 108 and the second sound generating assembly 128 are positioned adjacent to the respective ears of the user, maintained in position by the elastic head band 160. The first ear cushion 120 and the second ear cushion 140 are shown creating acoustic seals around the respective ear regions of the user. The first cover 124 and the second cover 144 of the respective sound generating assemblies 108, 128 are oriented in an outward-facing configuration, providing access to the power button 176 (as shown being pressed) and other control interfaces.

FIG. 2B illustrates the sleep mask 100 during active operation, with a representative audio waveform pattern generated by the sound generating assemblies 108, 128. The depicted waveform pattern represents the audio output characteristics of the first sound pattern, the second sound pattern, or the third sound pattern being generated by the first microprocessor 114 and transmitted through the first speaker 110 and the second speaker 130. The variable amplitude and frequency components of the waveform pattern correspond to the specific therapeutic sound sequences being executed, including the breath inhaling sounds, breath holding sounds, breath exhaling sounds, white noise, or wake-up alarm sounds as determined by the operational mode of the sleep mask 100.

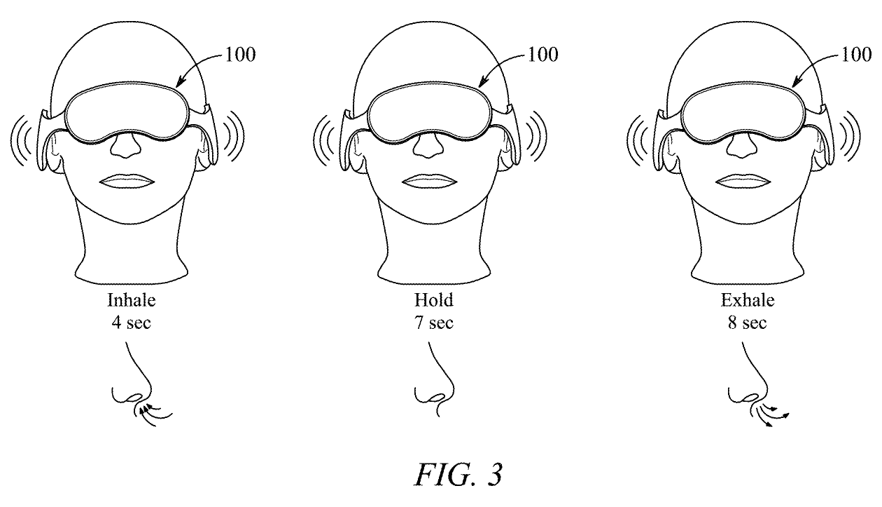

Referring to FIG. 3, illustrated is a sequence diagram depicting the three primary phases of the breathing guidance functionality implemented by the first sound pattern of the sleep mask 100. The sequence diagram includes three views, with each view corresponding to a specific phase of the breathing cycle and accompanied by a representative breathing airflow indicator. In the first phase, labeled “Inhale,” the first sound generating assembly 108 and the second sound generating assembly 128 generate synchronized audio guidance for a four-second inhalation period, indicated by radiating wave symbols and an inward-directed airflow arrow. The second phase, labeled “Hold,” depicts the seven-second breath retention period during which the sound generating assemblies 108, 128 provide audio cues for maintaining held breath, with the airflow indicator showing a neutral state. The third phase, labeled “Exhale,” depicts the eight-second exhalation period wherein the sound generating assemblies 108, 128 generate audio guidance for controlled breath release, depicted by the outward-directed airflow indicator. The complete nineteen-second breathing cycle is executed by the first microprocessor 114 according to timing parameters stored in the first programmable memory card 118, with the first speaker 110 and the second speaker 130 generating synchronized audio output to guide the user through each phase of the breathing sequence. This breathing cycle repeats three times within each five-minute interval during the initial twenty-minute operational period of the sleep mask 100.

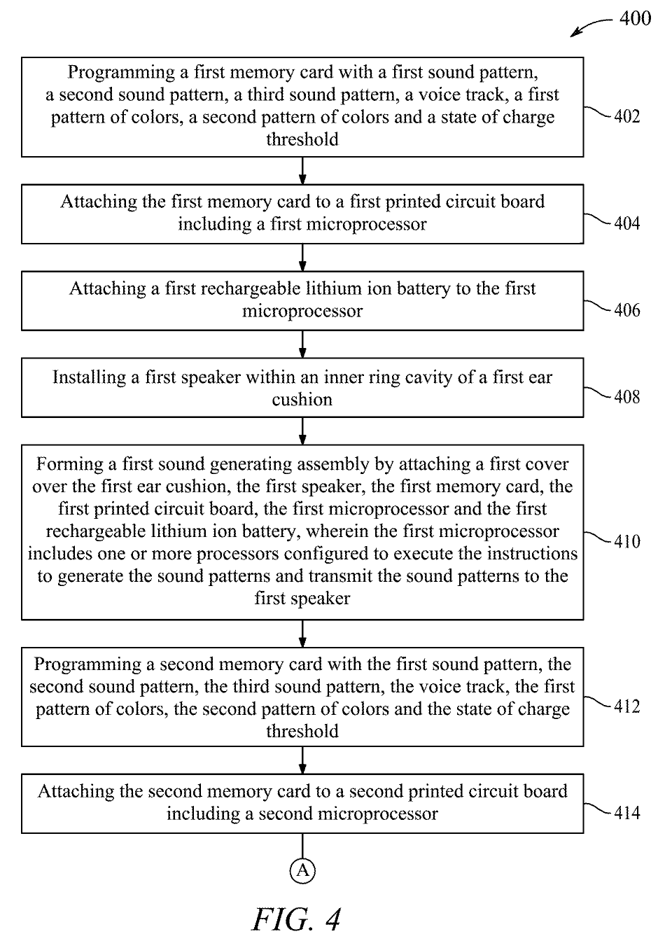

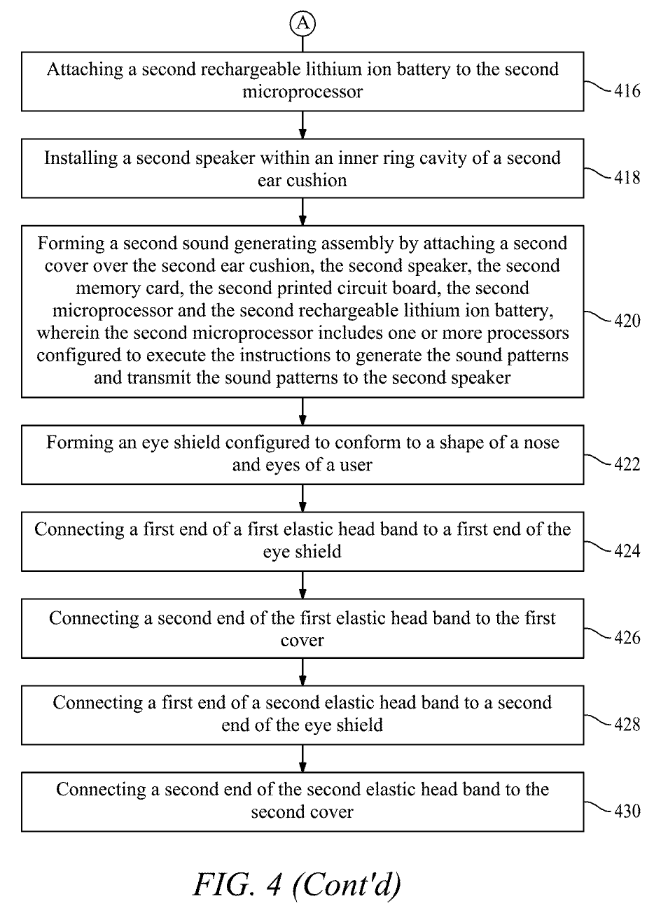

Referring now to FIG. 4, the present disclosure further provides a method (as represented by a flowchart, referred by reference numeral 400) of making the sleep mask 100. The method 400 includes a series of steps. These steps are only illustrative, and other alternatives may be considered where one or more steps are added, one or more steps are removed, or one or more steps are provided in a different sequence without departing from the scope of the present disclosure. Various variants disclosed above, with respect to the aforementioned sleep mask 100 apply mutatis mutandis to the present method 400.

At step 402, the method 400 includes programming the first memory card 118 with the first sound pattern, the second sound pattern, the third sound pattern, the voice track, the first pattern of colors, the second pattern of colors and a state of charge threshold. The method 400 includes programming the first programmable memory card 118 with multiple audio and visual patterns through a standard memory card interface. The first sound pattern is programmed to generate breathing sounds configured to guide a sequence of breaths of the user for a first period of time of twenty minutes when the power button 176 is turned ON. The breathing sounds include a breath inhaling sound generated for four seconds, a breath holding sound generated for seven seconds, and a breath exhaling sound generated for eight seconds, with this nineteen-second sequence repeating three times every five minutes. The voice track is programmed to provide synchronized counting sounds corresponding to each phase of the breathing sequence. The second sound pattern is programmed to generate calibrated white noise configured to activate automatically after the first period of time and continue until either manual deactivation through the power button 176 or expiration of a programmed sleep time. The third sound pattern is programmed to generate a wake-up alarm sound with gradually increasing volume when the programmed sleep time expires. The first pattern of colors and the second pattern of colors are programmed within specific frequency parameters to generate wavelengths within the red spectrum through the first light emitting diode 172 and the second light emitting diode 174 respectively. The state of charge threshold is programmed as a predetermined value for battery monitoring.

At step 404, the method 400 includes attaching the first memory card 118 to the first printed circuit board 112 including a first microprocessor 114. The first programmable memory card 118 is physically and electrically connected to the first printed circuit board 112 through a dedicated memory card connector mounted on the first printed circuit board 112. The mounting arrangement ensures secure mechanical attachment while maintaining reliable electrical connectivity between the first programmable memory card 118 and the first microprocessor 114.

At step 406, the method 400 includes attaching the first rechargeable lithium ion battery 116 to the first microprocessor 114. The first rechargeable lithium ion battery 116, rated at about 3000mAh capacity, is connected to the first printed circuit board 112 through dedicated power distribution contacts and mounting points. The first rechargeable lithium ion battery 116 is physically secured to maintain stable positioning while enabling power delivery to all electronic components within the first sound generating assembly 108 through conductive pathways on the first printed circuit board 112. The first lithium ion battery 116 is selected for its characteristics including lightweight design, long operational life, reasonable cost, and reliable electrical performance, which are desired for a portable sleep assistance device.

At step 408, the method 400 includes installing the first speaker 110 within the inner ring cavity 122 of the first ear cushion 120. The first speaker 110 is positioned within the first inner ring cavity 122 of the first ear cushion 120, with the first inner ring cavity 122 specifically dimensioned to create an enclosed acoustic chamber. The first ear cushion 120 comprises cotton material selected for optimal acoustic properties and user comfort, with the first inner ring cavity 122 incorporating specific geometrical parameters to enhance sound delivery while preventing external noise interference. The mounting arrangement establishes a sealed interface between the first speaker 110 and the first inner ring cavity 122 to prevent acoustic leakage while enabling proper sound transmission.

At step 410, the method 400 includes forming the first sound generating assembly 108 by attaching the first cover 124 over the first ear cushion 120, the first speaker 110, the first memory card 118, the first printed circuit board 112, the first microprocessor 114 and the first rechargeable lithium ion battery 116, wherein the first microprocessor 114 includes one or more processors configured to execute the instructions to generate the sound patterns and transmit the sound patterns to the first speaker 110. The first cover 124, fabricated from polyethylene plastic material, is configured to interlock with the first ear cushion 120. The first cover 124 incorporates raised tactile indicators adjacent to control interfaces and maintains water-resistant properties. The assembly process includes positioning of the first speaker 110, the first memory card 118, the first printed circuit board 112, the first microprocessor 114, and the first rechargeable lithium ion battery 116 within designated sections of the enclosure formed by the first cover 124 and the first ear cushion 120. The first microprocessor 114 maintains electrical connections to all components through conductive pathways on the first printed circuit board 112, enabling execution of stored program instructions to generate and transmit sound patterns to the first speaker 110.

At step 412, the method 400 includes programming the second memory card 138 with the first sound pattern, the second sound pattern, the third sound pattern, the voice track, the first pattern of colors, the second pattern of colors and the state of charge threshold. The second programmable memory card 138 is programmed with identical audio and visual patterns as the first memory card 118 through the standard memory card interface. The programming includes the breathing sound sequence parameters, white noise characteristics, wake-up alarm configurations, and color pattern specifications. The second programmable memory card 138 receives identical state of charge threshold values for battery monitoring purposes, ensuring synchronized operation with the first sound generating assembly 108.

At step 414, the method 400 includes attaching the second memory card 138 to the second printed circuit board 132 including the second microprocessor 134. The second programmable memory card 138 is mounted to the second printed circuit board 132 through a similar connector configuration as utilized in the first sound generating assembly 108. The second printed circuit board 132 may incorporate the same FR-4 material specifications and houses the pre-mounted second microprocessor 134. The mounting arrangement establishes electrical connectivity and mechanical stability characteristics as implemented in the first sound generating assembly 108.

At step 416, the method 400 includes attaching the second rechargeable lithium ion battery 136 to the second microprocessor 134. The second rechargeable lithium ion battery 136 is integrated with the second printed circuit board 132 utilizing similar power distribution architecture as implemented in the first sound generating assembly 108. The integration may include overcharge protection circuitry monitored by the second microprocessor 134. The second rechargeable lithium ion battery 136 may maintain consistent voltage levels across all electronic components within the second sound generating assembly 128.

At step 418, the method 400 includes installing the second speaker 130 within the inner ring cavity 142 of the second ear cushion 140. The second speaker 130 is mounted within the second inner ring cavity 142, maintaining dimensional specifications matching the first sound generating assembly 108. The cotton material composition of the second ear cushion 140 provides acoustic dampening and comfort characteristics. The mounting interface between the second speaker 130 and the second inner ring cavity 142 may also incorporate acoustic sealing features to prevent sound leakage.

At step 420, the method 400 includes forming the second sound generating assembly 128 by attaching the second cover 144 over the second ear cushion 140, the second speaker 130, the second memory card 138, the second printed circuit board 132, the second microprocessor 134 and the second rechargeable lithium ion battery 136, wherein the second microprocessor 134 includes one or more processors configured to execute the instructions to generate the sound patterns and transmit the sound patterns to the second speaker 130. The second cover 144 is formed of polyethylene plastic material with water-resistant properties and incorporates interlocking features matching the second ear cushion 140. The assembly process follows a component arrangement sequence ensuring proper alignment of the second speaker 130, the second memory card 138, the second printed circuit board 132, the second microprocessor 134, and the second rechargeable lithium ion battery 136. The second microprocessor 134 maintains electrical connectivity through the second printed circuit board 132, enabling synchronized operation with the first sound generating assembly 108 for coordinated audio output.

At step 422, the method 400 includes forming the eye shield 102 configured to conform to a shape of a nose and eyes of a user. The eye shield 102 is constructed through a multi-layer fabrication process incorporating a base layer of dust mite, mold, and mildew resistant cotton material overlaid with fade-resistant stretchable silk satin fabric. The eye shield 102 includes the contoured lower edge 104 to accommodate various facial structures while maintaining light-blocking properties. The multi-layer construction enables optimal breathability during extended wear periods while maintaining structural integrity.

At step 424, the method 400 includes connecting a first end of a first elastic head band to a first end of the eye shield 102. Herein, the first end of the first elastic head band is part of the first segment 162 of the elastic head band 160. The first segment 162 of the elastic head band 160 is integrated with the eye shield 102 through reinforced connection points. The attachment mechanism incorporates multi-point stitching patterns with tensile strength specifications optimized for repeated use cycles. The connection interface maintains specific elastic modulus characteristics to provide consistent pressure distribution across the eye shield 102 while preventing material fatigue during extended wear periods.

At step 426, the method 400 includes connecting a second end of the first elastic head band to the first cover 124. Herein, the second end of the first elastic head band is part of the second segment 164 of the elastic head band 160. The second segment 164 of the elastic head band 160 interfaces with the first cover 124 through reinforced connection points configured to maintain positional stability of the first sound generating assembly 108. The attachment mechanism provides controlled elastic deformation while maintaining secured positioning of the first sound generating assembly 108 relative to the ear region.

At step 428, the method 400 includes connecting a first end of a second elastic head band to a second end of the eye shield 102. Herein, the first end of the second elastic head band is part of the third segment 166 of the elastic head band 160. The third segment 166 of the elastic head band 160 integrates with the opposing end of the eye shield 102 utilizing identical reinforcement patterns and connection methodologies as implemented for the first segment 162. The attachment configuration maintains symmetric tension distribution characteristics across the eye shield 102 structure.

At step 430, the method 400 includes connecting a second end of the second elastic head band 168 to the second cover 144. Herein, the second end of the second elastic head band 168 is part of the fourth segment 168 of the elastic head band 160. The fourth segment 168 of the elastic head band 160 connects to the second cover 144 through attachment points matching the specifications utilized for the second segment 164 connection. The complete elastic head band assembly 160, comprising all four segments 162, 164, 166, 168, creates a unified tension distribution system maintaining optimal positioning of all components.

Herein, the first elastic head band and the second elastic head band configured to hold the eye shield 102 over the nose and eyes of the user, the first sound generating assembly 108 over a first ear of the user and the second sound generating assembly 128 over a second ear of the user. The integrated sleep mask 100 facilitates accurate spatial relationships between the eye shield 102 and both sound generating assemblies 108, 128, ensuring consistent acoustic seal formation around the ears while maintaining proper eye shield positioning. The elastic head band configuration accommodates head circumferences ranging from 52 centimeters to 62 centimeters through controlled elastic deformation characteristics, with specific tension parameters optimized for extended wear comfort and stability.

In an aspect, the method 400 further includes installing the power button 176 on the first cover 124, wherein the power button 176 is connected to the first rechargeable lithium ion battery 116 and the first microprocessor 114 of the first sound generating assembly 108. The power button 176 is integrated into the first cover 124, incorporating tactile feedback elements while maintaining the water-resistant properties of the first cover 124. The power button 176 establishes electrical connectivity through the first printed circuit board 112, interfacing with both the first rechargeable lithium ion battery 116 and the first microprocessor 114. The placement is specifically selected to allow easy access during use while preventing accidental activation.

In an aspect, the method 400 further includes generating, with the first sound pattern, a sequence of repetitious breathing sounds configured to guide a sequence of breaths of the user for a first period of time when the power button 176 is turned ON. Upon activation through the power button 176, the first microprocessor 114 initiates the first sound pattern comprising precisely timed breathing sequences. Each breathing cycle spans nineteen seconds, consisting of a breath inhaling sound for four seconds, a breath holding sound for seven seconds, and a breath exhaling sound for eight seconds. This sequence repeats three times within each five-minute interval throughout the initial twenty-minute operational period. The first microprocessor 114 implements synchronization between the first speaker 110 and the second speaker 130 to maintain coordinated audio output. The method 400 further includes generating, with the second sound pattern, white noise after the first period of time until one of the power button 176 is turned OFF and a programmed sleep time has expired. Following completion of the first sound pattern, the first microprocessor 114 transitions to generating calibrated white noise to mask ambient sounds and maintain sleep continuity. The white noise generation continues until either manual deactivation through the power button 176 or reaching the pre-programmed sleep duration threshold stored within the first programmable memory card 118. The method 400 further includes generating, with the third sound pattern, a wake-up alarm sound when the programmed sleep time has expired. Upon reaching the programmed sleep duration threshold, the first microprocessor 114 initiates the third sound pattern comprising a wake-up alarm sequence. The wake-up alarm implements gradually increasing volume levels to provide gentle arousal from sleep. The first microprocessor 114 maintains coordination between the first speaker 110 and second speaker 130 during the wake-up sequence, ensuring balanced stereo output.

In an aspect, the method 400 further includes generating the sequence of repetitious breathing sounds to include a breath inhaling sound generated for about four seconds, a breath holding sound generated for about seven seconds and a breath exhaling sound generated for about eight seconds. The first microprocessor 114 executes timing control for generating the three-phase breathing sequence. The breath inhaling sound is generated with a duration of 4000 milliseconds, followed by the breath holding sound maintained for 7000 milliseconds, and concluded with the breath exhaling sound extending for 8000 milliseconds. The timing parameters for each phase are controlled by the first microprocessor 114 according to specifications stored within the first programmable memory card 118, with the complete nineteen-second sequence repeated three times within each five-minute operational interval. The method 400 further includes generating, with the voice track, counting sounds as the sequence of repetitious breathing sounds are generated. The first microprocessor 114 provides synchronized playback of the voice track with the breathing sequence audio output. The voice track generates numerical counting sounds corresponding to each phase of the breathing sequence, with count synchronization maintained through timing parameters stored in the first programmable memory card 118.

In an aspect, the method 400 further includes installing the fiber optic fabric 170 within the eye shield 102 and within each elastic head band 160. The fiber optic fabric 170 is integrated within both the eye shield 102 and the elastic head band 160 through a network of optical fibers positioned to provide uniform illumination distribution. The installation process maintains specific fiber routing paths ensuring consistent light transmission characteristics throughout the integrated fabric structure. The fiber optic fabric 170 is configured to enable combination and interaction of multiple light patterns within the optical fibers. The method 400 further includes installing the first light emitting diode 172 on the first printed circuit board 112 and the second light emitting diode 174 on the second printed circuit board 132, wherein the first light emitting diode 172 is connected to the first microprocessor 114 and to the first plurality of optical fibers in the fiber optic fabric 170, and the second light emitting diode 174 is connected to the second microprocessor 134 and to the second plurality of optical fibers in the fiber optic fabric 170. The first light emitting diode 172 is electrically connected to the first microprocessor 114 and optically coupled to the first plurality of optical fibers, while the second light emitting diode 174 establishes equivalent connections with the second microprocessor 134 and the second plurality of optical fibers. This configuration enables synchronized illumination control through coordinated operation of both microprocessors. The method 400 further includes enhancing sleep during any one of the first sound pattern, the second sound pattern and both of the first sound pattern and the second sound pattern by actuating, by the microprocessor 114, 134, the light emitting diode 172, 174 of each sound generating assembly 108, 128 to illuminate the fiber optic fabric with colors in the red spectrum. The first microprocessor 114 and the second microprocessor 134 execute coordinated control algorithms to drive their respective light emitting diodes 172, 174 according to specific frequency patterns. The first microprocessor 114 generates the first pattern of colors within the red spectrum through the first light emitting diode 172, while the second microprocessor 134 generates the second pattern of colors within the red spectrum through the second light emitting diode 174. The light emitting diodes 172, 174 may additionally flicker during a first phase of the generation of the patterns of colors, in order to induce the patient to sleep. The fiber optic fabric 170 combines these patterns through interaction within the optical fibers to produce wavelengths demonstrated to enhance sleep quality. The timing and intensity parameters for both patterns are programmed into the respective memory cards 118, 138 and coordinated by the microprocessors 114, 134 to maintain optimal illumination characteristics throughout the sleep cycle. In an aspect, the

The sleep mask 100 and the method 400 of the present disclosure provide an integrated therapeutic solution through synchronization of multiple sleep-inducing mechanisms within a unified structural framework. The sleep mask 100 implements coordinated delivery of breathing guidance, white noise generation, and spectrum-specific illumination through precisely controlled electronic systems. The sleep mask 100 incorporates dual sound generating assemblies 108, 128 operating in synchronized configurations to deliver three distinct therapeutic sound patterns, while simultaneously providing controlled illumination through an integrated fiber optic fabric 170 activated by coordinated light emitting diodes 172, 174 generating specific wavelengths within the red spectrum demonstrated to enhance sleep quality. The sleep mask 100 overcomes limitations of conventional sleep masks through implementation of programmable therapeutic sequences tailored to user sleep requirements. This coordinated delivery of multiple therapeutic modalities provides comprehensive sleep assistance functionality exceeding capabilities of single-mode therapeutic devices.

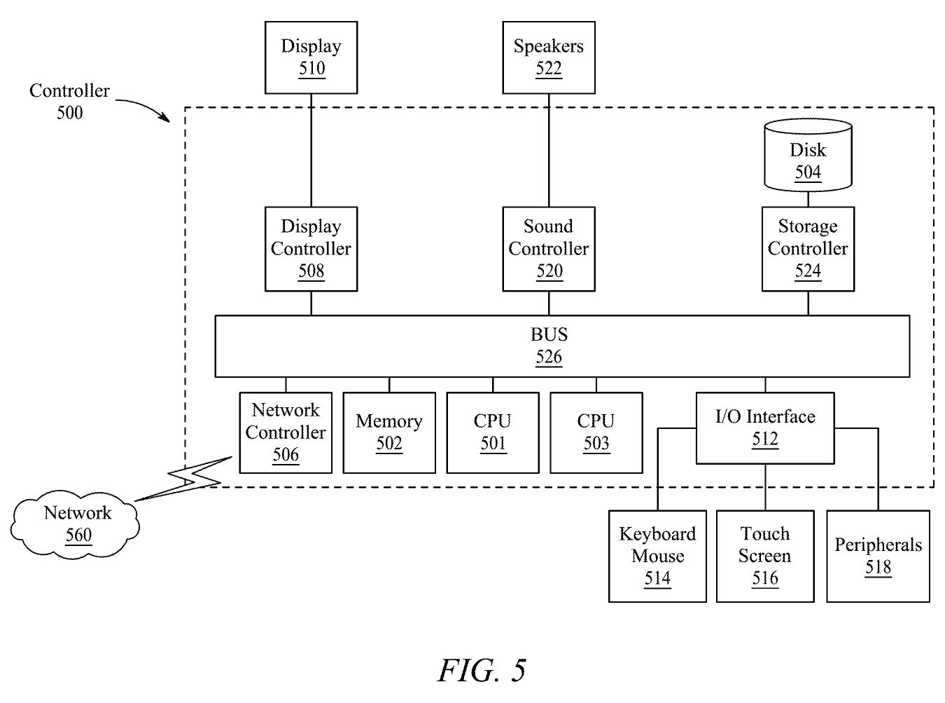

Next, further details of the hardware description of a computing environment according to exemplary embodiments is described with reference to FIG. 5. In FIG. 5, a controller 500 is described is representative of the microprocessors 114, 134 of the present sleep mask 100 to control operations of various electrical components therein, in which the controller 500 is a computing device which includes a CPU 501 which performs the processes described above/below. The process data and instructions may be stored in memory 502. These processes and instructions may also be stored on a storage medium disk 504 such as a hard drive (HDD) or portable storage medium or may be stored remotely.

Further, the claims are not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the claims may be provided as a utility application, background daemon, or component of an operating system, or combination thereof, executing in conjunction with CPU 501, 503 and an operating system such as Microsoft Windows 7, Microsoft Windows 8, Microsoft Windows 10, UNIX, Solaris, LINUX, Apple MAC-OS and other systems known to those skilled in the art.

The hardware elements in order to achieve the computing device may be realized by various circuitry elements, known to those skilled in the art. For example, CPU 501 or CPU 503 may be a Xenon or Core processor from Intel of America or an Opteron processor from AMD of America, or may be other processor types that would be recognized by one of ordinary skill in the art. Alternatively, the CPU 501, 503 may be implemented on an FPGA, ASIC, PLD or using discrete logic circuits, as one of ordinary skill in the art would recognize. Further, CPU 501, 503 may be implemented as multiple processors cooperatively working in parallel to perform the instructions of the inventive processes described above.

The computing device in FIG. 5 also includes a network controller 506, such as an Intel Ethernet PRO network interface card from Intel Corporation of America, for interfacing with network 560. As can be appreciated, the network 560 can be a public network, such as the Internet, or a private network such as an LAN or WAN network, or any combination thereof and can also include PSTN or ISDN sub-networks. The network 560 can also be wired, such as an Ethernet network, or can be wireless such as a cellular network including EDGE, 3G, 4G and 5G wireless cellular systems. The wireless network can also be WiFi, Bluetooth, or any other wireless form of communication that is known.

The computing device further includes a display controller 508, such as a NVIDIA GeForce GTX or Quadro graphics adaptor from NVIDIA Corporation of America for interfacing with display 510, such as a Hewlett Packard HPL2445w LCD monitor. A general purpose I/O interface 512 interfaces with a keyboard and/or mouse 514 as well as a touch screen panel 516 on or separate from display 510. General purpose I/O interface also connects to a variety of peripherals 518 including printers and scanners, such as an OfficeJet or DeskJet from Hewlett Packard.

A sound controller 520 is also provided in the computing device such as Sound Blaster X-Fi Titanium from Creative, to interface with speakers/microphone 522 thereby providing sounds and/or music.

The general purpose storage controller 524 connects the storage medium disk 504 with communication bus 526, which may be an ISA, EISA, VESA, PCI, or similar, for interconnecting all of the components of the computing device. A description of the general features and functionality of the display 510, keyboard and/or mouse 514, as well as the display controller 508, storage controller 524, network controller 506, sound controller 520, and general purpose I/O interface 512 is omitted herein for brevity as these features are known.

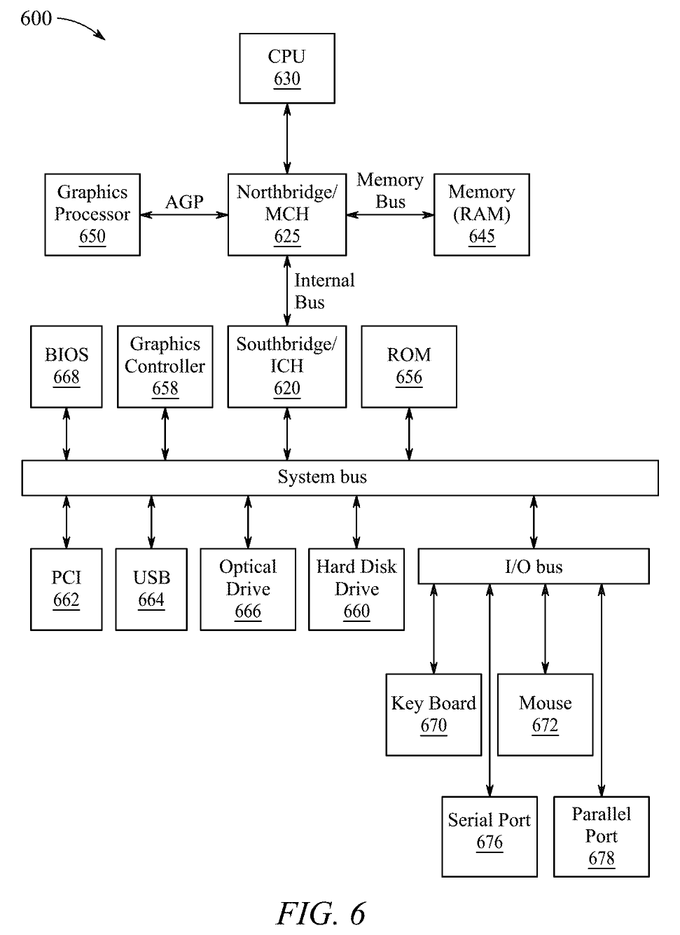

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 6.

FIG. 6 shows a schematic diagram of a data processing system, according to certain embodiments, for performing the functions of the exemplary embodiments. The data processing system is an example of a computer in which code or instructions implementing the processes of the illustrative embodiments may be located.

In FIG. 6, data processing system 600 employs a hub architecture including a north bridge and memory controller hub (NB/MCH) 625 and a south bridge and input/output (I/O) controller hub (SB/ICH) 620. The central processing unit (CPU) 630 is connected to NB/MCH 625. The NB/MCH 625 also connects to the memory 645 via a memory bus, and connects to the graphics processor 650 via an accelerated graphics port (AGP). The NB/MCH 625 also connects to the SB/ICH 620 via an internal bus (e.g., a unified media interface or a direct media interface). The CPU Processing unit 630 may contain one or more processors and even may be implemented using one or more heterogeneous processor systems.

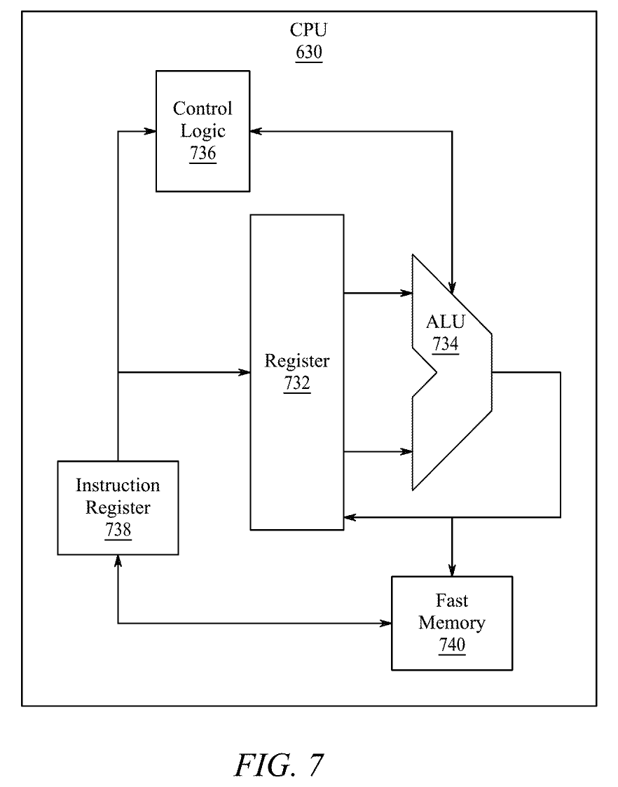

For example, FIG. 7 shows one implementation of CPU 630. In one implementation, the instruction register 738 retrieves instructions from the fast memory 740. At least part of these instructions are fetched from the instruction register 738 by the control logic 736 and interpreted according to the instruction set architecture of the CPU 630. Part of the instructions can also be directed to the register 732. In one implementation the instructions are decoded according to a hardwired method, and in another implementation the instructions are decoded according a microprogram that translates instructions into sets of CPU configuration signals that are applied sequentially over multiple clock pulses. After fetching and decoding the instructions, the instructions are executed using the arithmetic logic unit (ALU) 734 that loads values from the register 732 and performs logical and mathematical operations on the loaded values according to the instructions. The results from these operations can be feedback into the register and/or stored in the fast memory 740. According to certain implementations, the instruction set architecture of the CPU 630 can use a reduced instruction set architecture, a complex instruction set architecture, a vector processor architecture, a very large instruction word architecture. Furthermore, the CPU 630 can be based on the Von Neuman model or the Harvard model. The CPU 630 can be a digital signal processor, an FPGA, an ASIC, a PLA, a PLD, or a CPLD. Further, the CPU 630 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 6, the data processing system 600 can include that the SB/ICH 620 is coupled through a system bus to an I/O Bus, a read only memory (ROM) 656, universal serial bus (USB) port 664, a flash binary input/output system (BIOS) 668, and a graphics controller 658. PCI/PCIe devices can also be coupled to SB/ICH 688 through a PCI bus 662.

The PCI devices may include, for example, Ethernet adapters, add-in cards, and PC cards for notebook computers. The Hard disk drive 660 and CD-ROM 666 can use, for example, an integrated drive electronics (IDE) or serial advanced technology attachment (SATA) interface. In one implementation the I/O bus can include a super I/O (SIO) device.

Further, the hard disk drive (HDD) 660 and optical drive 666 can also be coupled to the SB/ICH 620 through a system bus. In one implementation, a keyboard 670, a mouse 672, a parallel port 678, and a serial port 676 can be connected to the system bus through the I/O bus. Other peripherals and devices that can be connected to the SB/ICH 620 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

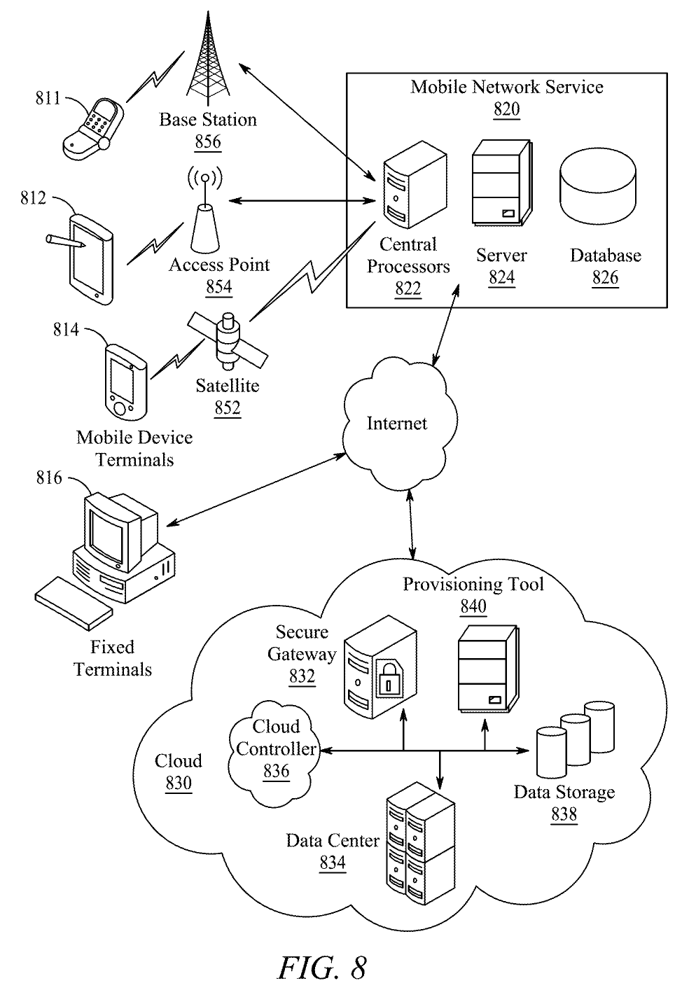

The functions and features described herein may also be executed by various distributed components of a system. For example, one or more processors may execute these system functions, wherein the processors are distributed across multiple components communicating in a network. The distributed components may include one or more client and server machines, such as cloud 830 including a cloud controller 836, a secure gateway 832, a data center 834, data storage 838 and a provisioning tool 840, and mobile network services 820 including central processors 822, a server 824 and a database 826, which may share processing, as shown by FIG. 8, in addition to various human interface and communication devices (e.g., display monitors 816, smart phones 810, tablets 812, personal digital assistants (PDAs) 814). The network may be a private network, such as a LAN, satellite 852 or WAN 854, or be a public network, may such as the Internet. Input to the system may be received via direct user input and received remotely either in real-time or as a batch process. Additionally, some implementations may be performed on modules or hardware not identical to those described. Accordingly, other implementations are within the scope that may be claimed.

While specific embodiments of the invention have been described, it should be understood that various modifications and alternatives may be implemented without departing from the spirit and scope of the invention. For example, different cellular automata rules or encryption algorithms could be employed, or alternative feature extraction and face recognition techniques could be integrated into the system.

The above-described hardware description is a non-limiting example of corresponding structure for performing the functionality described herein.

Numerous modifications and variations of the present disclosure are possible in light of the above teachings. It is therefore to be understood that within the scope of the appended claims, the invention may be practiced otherwise than as specifically described herein.

1. A sleep mask, comprising:
an eye shield configured to conform to a shape of a nose and eyes of a user;
a first sound generating assembly including a first speaker;
a second sound generating assembly including a second speaker, wherein each sound generating assembly includes a printed circuit board, an ear cushion configured with an inner ring cavity and a cover, wherein the cover is configured to interlock with the ear cushion to enclose the printed circuit board, wherein the first speaker is configured to fit into the inner ring cavity of the ear cushion of the first sound generating assembly and the second speaker is configured to fit into the inner ring cavity of the ear cushion of the second sound generating assembly; and
an elastic head band including a first segment connected to a proximal edge of the ear cushion of the first sound generating assembly and a second segment connected to a distal edge of the ear cushion of the first sound generating assembly, a third segment connected to a proximal edge of the ear cushion of the second sound generating assembly and a fourth segment connected to a distal edge of the ear cushion of the second sound generating assembly, wherein the elastic head band is configured to hold the eye shield over the nose and eyes of the user and the sound generating assembly over each ear of the user.

2. The sleep mask of claim 1, wherein each sound generating assembly further comprises:
a rechargeable lithium ion battery connected to the printed circuit board;
a programmable memory card connected to the printed circuit board, wherein the programmable memory card includes program instructions which include sound patterns;
a microprocessor connected to the printed circuit board, the programmable memory card, the rechargeable lithium ion battery, and the speaker, wherein the microprocessor includes one or more processors configured to execute the instructions to generate the sound patterns and transmit the sound patterns to the speaker.

3. The sleep mask of claim 2, wherein the microprocessor of the first sound generating assembly is configured to generate the sound patterns and transmit the sound patterns to the speaker of the first sound generating assembly and to the speaker of the second sound generating assembly, wherein the speaker of the first sound generating assembly and the speaker of the second sound generating assembly generate the sound patterns in tandem.

4. The sleep mask of claim 2, further comprising:
a power button located on the cover of the first sound generating assembly, wherein the power button is connected to the rechargeable lithium ion battery and the microprocessor of the first sound generating assembly.

5. The sleep mask of claim 2, further comprising:
an indicator light located on the cover of each sound generating assembly, wherein the microprocessor of each sound generating assembly is configured to determine a state of charge of the rechargeable lithium ion battery, compare the state of charge to a threshold value and illuminate the indicator light when the state of charge is less than the threshold value.

6. The sleep mask of claim 5, further comprising:
a first charge jack connected to the cover of the first sound generating assembly, wherein the first charge jack is configured to connect to a shared charger to recharge the rechargeable lithium ion battery of the first sound generating assembly; and
a second charge jack connected to the cover of the second sound generating assembly, wherein the second charge jack is configured to connect to the shared charger to recharge the rechargeable lithium ion battery of the second sound generating assembly.

7. The sleep mask of claim 2, wherein the sound patterns of each sound generating assembly comprise:
a first sound pattern configured to generate breathing sounds to guide a sequence of breaths of the user for a first period of time when the power button is turned ON; and
a second sound pattern configured to generate white noise after the first period of time until one of the power button is turned OFF and a programmed sleep time has expired; and
a third sound pattern configured to generate a wake-up alarm sound when the programmed sleep time has expired.

8. The sleep mask of claim 7, wherein the first sound generating assembly further comprises:
a set of alarm control buttons located on the cover and connected to the microprocessor; and
a clock timer located within the microprocessor,
wherein the set of alarm control buttons are configured to program the microprocessor to generate the wake-up alarm sound of the third sound pattern.

9. The sleep mask of claim 7, wherein the first sound pattern comprises:
a sequence of repetitious breathing sounds configured to repeat three times every five minutes, wherein the sequence of repetitious breathing sounds includes a breath inhaling sound generated for about four seconds, a breath holding sound generated for about seven seconds and a breath exhaling sound generated for about eight seconds.

10. The sleep mask of claim 9, wherein the first sound pattern of each sound generating assembly further comprises a voice track configured to provide counting sounds as the sequence of repetitious breathing sounds are generated.

11. The sleep mask of claim 9, wherein the first period of time is about twenty minutes.

12. The sleep mask of claim 7, further comprising:
a fiber optic fabric located within the eye shield and within the elastic head band; and
a light emitting diode located on the printed circuit board of each sound generating assembly, wherein the light emitting diode is connected to the microprocessor and to a plurality of optical fibers in the fiber optic fabric, wherein each microprocessor is configured to actuate the light emitting diode to illuminate the fiber optic fabric in colors in a red spectrum configured to enhance sleep during any one of the first sound pattern, the second sound pattern and both of the first sound pattern and the second sound pattern.

13. The sleep mask of claim 12, wherein the microprocessor of the first sound generating assembly is configured to drive the light emitting diode in a first frequency pattern to generate a first pattern of colors and the microprocessor of the second sound generating assembly is configured to drive the light emitting diode in a second frequency pattern to generate a second pattern of colors, wherein the first pattern of colors and the second pattern of colors mix within the fiber optic fibers to provide a new color pattern which is configured to enhance sleep.

14. The sleep mask of claim 2, wherein the cover, the memory card and an enclosure of the speaker of each sound generating assembly are made of polyethylene and the printed circuit board is made of polychlorinated biphenyl plastic.

15. The sleep mask of claim 1, wherein the eye shield is made of a layer of dust mite, mold, and mildew resistant cotton covered by a fade resistant stretchable silk satin fabric.

16. A method of making a sleep mask, comprising:
programming a first memory card with a first sound pattern, a second sound pattern, a third sound pattern, a voice track, a first pattern of colors, a second pattern of colors and a state of charge threshold;
attaching the first memory card to a first printed circuit board including a first microprocessor;
attaching a first rechargeable lithium ion battery to the first microprocessor;
installing a first speaker within an inner ring cavity of a first ear cushion;
forming a first sound generating assembly by attaching a first cover over the first ear cushion, the first speaker, the first memory card, the first printed circuit board, the first microprocessor and the first rechargeable lithium ion battery, wherein the first microprocessor includes one or more processors configured to execute the instructions to generate the sound patterns and transmit the sound patterns to the first speaker;
programming a second memory card with the first sound pattern, the second sound pattern, the third sound pattern, the voice track, the first pattern of colors, the second pattern of colors and the state of charge threshold;
attaching the second memory card to a second printed circuit board including a second microprocessor;
attaching a second rechargeable lithium ion battery to the second microprocessor;
installing a second speaker within an inner ring cavity of a second ear cushion;
forming a second sound generating assembly by attaching a second cover over the second ear cushion, the second speaker, the second memory card, the second printed circuit board, the second microprocessor and the second rechargeable lithium ion battery, wherein the second microprocessor includes one or more processors configured to execute the instructions to generate the sound patterns and transmit the sound patterns to the second speaker;
forming an eye shield configured to conform to a shape of a nose and eyes of a user;
connecting a first end of a first elastic head band to a first end of the eye shield;
connecting a second end of the first elastic head band to the first cover; and
connecting a first end of a second elastic head band to a second end of the eye shield;
connecting a second end of the second elastic head band to the second cover,
wherein the first elastic head band and the second elastic head band configured to hold the eye shield over the nose and eyes of the user, the first sound generating assembly over a first ear of the user and the second sound generating assembly over a second ear of the user.

17. The method of claim 16, further comprising:
installing a power button on the first cover, wherein the power button is connected to the rechargeable lithium ion battery and the microprocessor of the first sound generating assembly.

18. The method of claim 17, further comprising:
generating, with the first sound pattern, a sequence of repetitious breathing sounds configured to guide a sequence of breaths of the user for a first period of time when the power button is turned ON; and
generating, with the second sound pattern, white noise after the first period of time until one of the power button is turned OFF and a programmed sleep time has expired; and
generating, with the third sound pattern, a wake-up alarm sound when the programmed sleep time has expired.

19. The method of claim 18, further comprising:
generating the sequence of repetitious breathing sounds to include a breath inhaling sound generated for about four seconds, a breath holding sound generated for about seven seconds and a breath exhaling sound generated for about eight seconds; and
generating, with the voice track, counting sounds as the sequence of repetitious breathing sounds are generated.

20. The method of claim 19, further comprising:
installing a fiber optic fabric within the eye shield and within each elastic head band; and
installing a light emitting diode on the printed circuit board of each sound generating assembly, wherein the light emitting diode is connected to the microprocessor and to a plurality of optical fibers in the fiber optic fabric;
enhancing sleep during any one of the first sound pattern, the second sound pattern and both of the first sound pattern and the second sound pattern by actuating, by the microprocessor, the light emitting diode of each sound generating assembly to illuminate the fiber optic fabric with colors in a red spectrum.
